(19) United States
(12) Patent Application Publication
(10) Pub. No.: US 2003/0017568 A1
(43) Pub. Date: Jan. 23, 2003

(54) Smooth muscle myosin phosphatase associated kinase

(76) Inventor: Timothy A. Haystead, Chapel Hill, NC (US)

Correspondence Address: NIXON & VANDERHYE P.C., 1100 North Glebe Road, 8th Floor, Arlington, VA, 22201, US

(21) Appl. No.: 10/083,641

(22) Filed: Feb. 27, 2002

Related U.S. Application Data
(60) Provisional application No. 60/271,436, filed on Feb. 27, 2001.

(51) Int. Cl. C12N 9/12; C07H 21/04; C12P 21/02; C12N 5/06

(52) U.S. Cl. 435/194; 435/69.1; 435/320.1; 435/325; 536/23.2

(57) ABSTRACT

The present invention relates to a novel smooth muscle myosin phosphate associated kinase and to methods of identifying compounds useful in treating smooth muscle disease using same.

This application claims priority from Provisional Application No. 60/271,436, filed Feb. 27, 2001, the entire content of which is incorporated herein by reference.

### TECHNICAL FIELD

The present invention relates to a novel smooth muscle myosin phosphate associated kinase and to methods of identifying compounds useful in treating smooth muscle disease using same.

### BACKGROUND

The major mechanism (Hartshorne in Physiology of the Gastrointestinal Tract, ed. Johnson, L. R. (Raven Press, New York, N.Y.), pp. 423-482 (1987), Sellers et al, Curr. Top. Cell. Regul. 27:51-62 (1985), Somlyo et al in The Heart and Cardiovascular System, ed. Fozzard, H. A. (Raven Press, New York, N.Y., pp. 1-30 (1991)) linking transients in [Ca²⁺]ᵢ to force in smooth muscle is by phosphorylation of the 20 kDa myosin light chain (MLC20). The level of phosphorylated myosin is controlled by two enzymes: a Ca²⁺-calmodulin dependent myosin light chain kinase (MLCK) and a myosin light chain phosphatase (SMPP-1M) (Somlyo et al, Nature 372:231-236 (1994), Hartshorne et al, J. Muscle Res. Cell. Motil. 19:325-341 (1998)). However, at fixed Ca²⁺levels contraction can also be induced by agonist stimulation or by activation of G-proteins with GTPγS or AlF₄ (Somlyo et al, Nature 372:231-236 (1994)). This leads to so-called Ca²⁺-sensitization (Somlyo et al, Nature 372:231-236 (1994), Hartshorne et al, J. Muscle Res. Cell. Motil. 19:325-341 (1998), Nishimura et al, Adv. in Exp. Med. Biol. 308:9-25 (1991), Kitazawa et al, J. Biol. Chem. 266:1708-1715 (1991)) and was shown to reflect an inhibition of SMPP-1M activity (Somlyo et al, Nature 372:231-236 (1994), Hartshorne et al, J. Muscle Res. Cell. Motil. 19:325-341 (1998). Somlyo et al, Adv. Protein Phosphatases 5:181-195 (1989), Kimura et al, Science 273:245-248 (1996)).

Protein phosphatase 1 (PP-1) is one of the major Ser/Thr protein phosphatases in eukaryotic cells, and different forms of PP-1 are composed of a catalytic subunit and different regulatory subunits that target the phosphatase to specific locations and particular substrates (Alms et al, EMBO J. 18:4157-4168 (1999), Hubbard et al, Trends Biochem. Sci. 18:172-177 (1993), Egloff et al, EMBO J. 16:1876-1887 (1997)). SMPP-IM is composed of three subunits: the 37 kDa catalytic subunit of PP-1 (PP1Cδ); a 110-130 kDa regulatory myosin phosphatase targeting subunit (MYPT1) and a 20 kDa subunit of undetermined function (Shirazi et al, J. Biol. Chem. 269:31598-31606 (1994), Alessi et al, Eur. J. Biochem. 210:1023-1035 (1992), Shimizu et al, J. Biol. Chem. 269:30407-30411 (1994)). The myosin phosphatase activity of SMPP-1M is thought to be regulated by phosphorylation of the MYPT1 subunit. There are several phosphorylation sites on MYPT1 including an inhibitory site of phosphorylation by an endogenous kinase (Ichikawa et al, J. Biol. Chem. 271:4733-4740 (1996)) identified as Thr⁶⁹⁵ (in the chicken MYPT1 isoform). Subsequent data indicated that there are two major sites on MYPT1 for Rho-associated protein kinase (ROK). These are Thr⁶⁹⁷ (numbering for rat isoform and equivalent to Thr⁶⁹⁵) and Ser⁸⁵⁴ (Kimura et al, Science 273:245-248 (1996), Kawano et al, J. Cell. Biol. 147:1023-1038 (1999), Feng et al, J. Biol. Chem. 274:37385-37390 (1999)). Recently it was shown that the site responsible for inhibition of SMPP-1M is Thr⁶⁹⁷ (Feng et al, J. Biol. Chem. 274:37385-37390 (1999)). Thus, it is clear that ROK plays an important role in Ca²⁺-sensitization of smooth muscle. However, the finding of an additional endogenously associated MYPT1 kinase (Ichikawa et al, J. Biol. Chem. 271:4733-4740 (1996)) and the recruitment of ROK to RhoA-GTP at the cell membrane raises both temporal and spatial concerns about access of ROK to the substrate MYPT1. The present invention results from a study designed to clarify this situation and to identify the endogenous or SMPP-1M associating kinase.

### BRIEF DESCRIPTION OF THE DRAWINGS

FIGS. 1A, B, c. Determination of the sites of phosphorylation of MYPT1 in vivo. A. ³²P-orthophosphate labeled rabbit bladder was stimulated with 10 μM carbachol for the indicated times. MYPT1 was immunoprecipitated from tissue homogenates then resolved by SDS-PAGE. Increased MYPT1 phosphorylation was determined by autoradiography. B. MYPT1 immunoprecipitated from control and carbachol stimulated rabbit bladder was digested overnight with trypsin; the ³²P labeled peptides obtained were separated on a C18 reverse phase column and identified by scintillation counting. C. One of the phosphorylated MYPT1 peptides (#2) was sequenced and its phosphorylation site identified as described (Ishizaki et al, EMBO J. 15:1885-1893 (1996)).

FIGS. 2, A, B. Endogenous kinase copurifies with SMPP-1M. Autoradiography (Inset A) of purified SMPP-1M shows a phosphorylated band at 110 kDa, correlating with MYPT1. SMPP-1M was affinity purified as described (Shirazi et al, J. Biol. Chem. 269:31598-31606 (1994)) and the purified enzyme incubated with 100 μM γ-[³²P] ATP and 2 mM MgCl₂. The reaction was terminated with sample buffer and MYPT1 resolved on SDS-PAGE gels. B. Purified M110 kinases accelerates the rate of SMPP-1M inactivation in vitro. Purified SMPP-1M was incubated for the indicated times with Mg/ATP (2 mM/100 μM) in the presence (◯) or absence (●) of affinity purified M110 kinase. Note: inactivation of SMPP-1M in the absence of exogenously added M110 kinase was due to the presence of endogenous copurifying kinase activity. A. The myofibrilar extract from rabbit bladder was resolved on an AP-1Q (0.5×7 cm) anion exchange column; the column was developed with a 0-1M NaCl gradient. SMPP-1M (◯) was assayed against ³²P labeled myosin and SMPP-1M kinase activity (●) was assayed against the Thr⁶⁹⁷ substrate peptide (KKKRQSRRSTQGVTL).

FIG. 3A, b-d. Purification of SMPP-1M associated kinase. A. SMPP-1M kinase was eluted from a Smart MiniQ (1.6/5) anion exchange column with a 0-1M NaCl gradient and identified using both in vitro and in gel kinase assay. The autoradiogram, inset b, of the in gel assay localized kinase activity to a discrete protein band at 32 kDa. Inset c is the results obtained from phosphoamino acid analysis (Feng et al, J. Biol. Chem. 24:3744-3752 (1999)) of Thr⁶⁹⁷ substrate peptide phosphorylated during the in vitro assay by purified SMPP-1M kinase. Phosphorylated Thr⁶⁹⁷ substrate peptide was sequenced and its phosphorylation site determined as described (Ishizaki et al, EMBO J. 15:1885-1893 (1996)). Inset d In-gel kinase assay comparing purified PKA (1 μg) control with purified SMPP-1M kinase. A control gel run in the absence of Thr⁶⁹⁷ substrate peptide was blank.

FIG. 4. Identification of SMPP-1M associated kinase by mixed peptide sequencing. Mixed sequence is listed in order of the PTH amino acids recovered after each Edman cycle. Sequence data shown was derived from 200 fmol of protein. FASTF was used to search and match the mixed sequences to the NCBI/Human protein database. The scoring matrix was MD20, with expectation and score values set to <1 and 5, respectively (Kameshita et al, Anal. Biochem. 183:139-143 (1989)). The highest scoring proteins were human ZIPK, (e) 5.1 e-14; human pDAPK3, (e) 5.1 e-14; and rat DAP-like kinase, (e) 2.1 e-7. The next highest unrelated protein score was D-glycerate dehydrogenase, (e) 0.0011.

FIGS. 5A-D. ZIP-like-kinase properties toward MYPT1. A. Effect of ROK inhibitor Y-27632 on ZIPK and ZIP-like-kinase. Kinases were assayed in vitro against the Thr⁶⁹⁷ peptide. B. Substrate concentration dependence of purified bladder ZIP kinase (◯), and ROK (●). Inset c, Autoradiograms showing phosphorylation of chicken gizzard full length MYPT1 (Feng et al, J. Biol. Chem. 274:37385-37390 (1999)), rM133, and chicken gizzard C-terminal fragment (Inbal et al, Mol. Cell. Biol. 20:1044-1054 (2000)), C130⁵¹⁴⁻⁹⁶³, by purified bladder ZIPK and ROK in vitro. Data are means ±SEM of three separate experiments. Inset d, Identification of the autophosphorylation sites on ZIPK.

FIGS. 6A-C, d, e. Association of SMPP-1M with ZIP-like-kinase. A. MYPT1 was immunoprecipitated and ZIP-like-kinase measured in the immunoprecipitate. Alternatively, ZIP-like-kinase was immunoprecipitated and myosin phosphatase measured against B. glycogen phosphorylase a or C. myosin. Inset D, tissue extracts from bladder were immunoprecipitated with anti-MYPT1 antibody, resolved on SDS-PAGE and immunoblotted for ZIPK. Inset E, tissue extracts from bladder were immunoprecipitated with anti-ZIPK antibody, resolved on SDS-PAGE and immunoblotted for MYPT1.

FIGS. 7a-c. Carbachol affects ZIP-like-kinase phosphorylation and activity in smooth muscle. [³²P] orthophosphate labeled rabbit bladder was stimulated with 50 μM carbachol in the presence of 10 μM calyculin A. Triton-extracted tissue pellets were fractionated on a SMART MiniQ (1.6/5 cm) column. A. Aliquots of fractions were run on SDS-PAGE gels and subjected to autoradiography (inset b) to visualize phosphorylation. Western immunoblots were used to identify the protein bands that corresponded with ZIPK. SMART fractions from both control (C) and carbachol (T) treated bladder containing ZIP-like-kinase were pooled, immunoprecipitated with anti-ZIP kinase antibody, and resolved on SDS-PAGE prior to autoradiography (inset b). B. Carbachol/calyculin A treatment increase ZIP-like-kinase activity. Homogenates were prepared and MYPT1 was immunoprecipitated. Immunoprecipitates were assessed in duplicate for ZIP-like-kinase activity. Activity shown was derived following subtraction of non-specific background kinase activity that was also present in the immunoprecipitate. Data represent the means ±SEM of five separate experiments, *-significantly different from the control value by the Student-Newman-Keuls test, p<0.05; **-significantly different from the carbachol/calyculin A treatment, p<0.05.

FIG. 8. Putative nucleotide sequence of the smooth muscle MYPT-kinase showing start site in bold.

FIG. 9. Deduced amino acid sequence of the rat aorta smooth muscle MYPT kinase (underlined shows alignment with 52 kDa ZIP kinase sequence)

### DETAILED DESCRIPTION OF THE INVENTION

It has been shown that the holoenzyme of myosin phosphatase co-purifies with an endogenous kinase that phosphorylates the MYPT1 subunit and inhibits phosphatase activity (Ichikawa et al, J. Biol. Chem. 271:4733-4740 (1996)). However, the identity of the kinase was unknown until the development of specialized affinity chromatography media and advances in protein microsequencing. With these techniques, it has been possible to purify a 32 kDa protein kinase that was identified by mixed peptide sequencing to be similar to HeLa zipper interacting protein kinase (ZIP kinase). Further in-gel kinase analysis by 2D SDS PAGE and mixed peptide sequencing confirmed that the 32 kDa band contained a single protein, MYPT-kinase, and not any other protein kinase. A previous report (Kawai et al, Mol. Cell. Biol. 18:1642-1651 (1998)) on full-length mammalian ZIP kinases indicated masses of 51.4 kDa and 52.5 kDa for the mouse and human isoforms, respectively, as compared to a mass of 32 kDa for the smooth muscle MYPT-kinase identified herein.

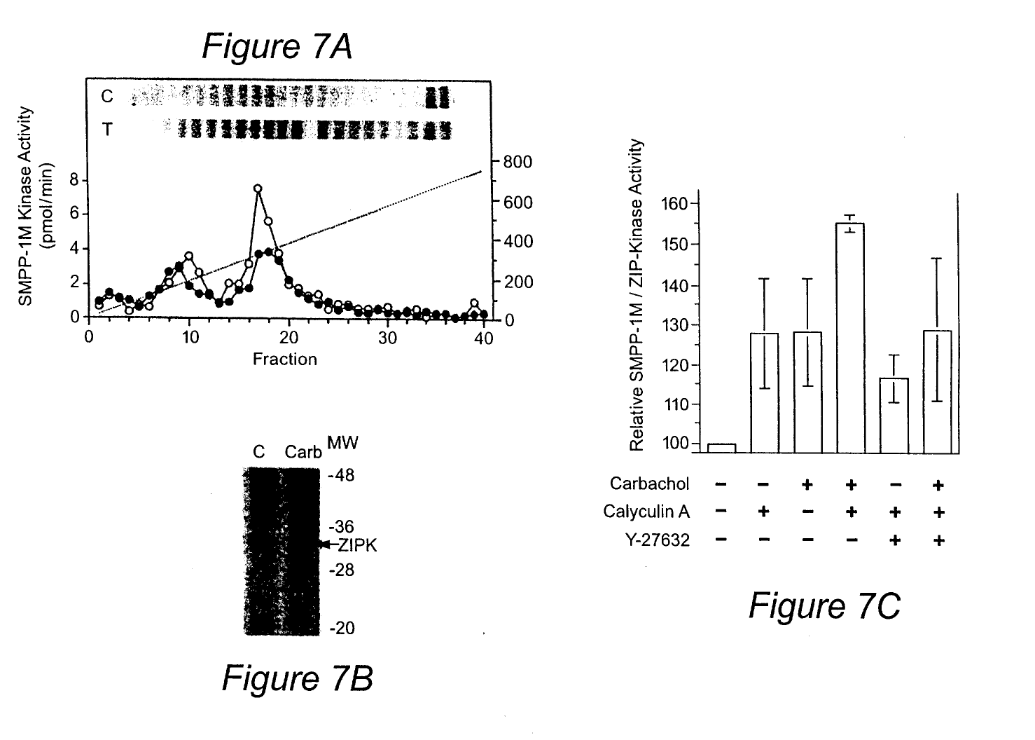

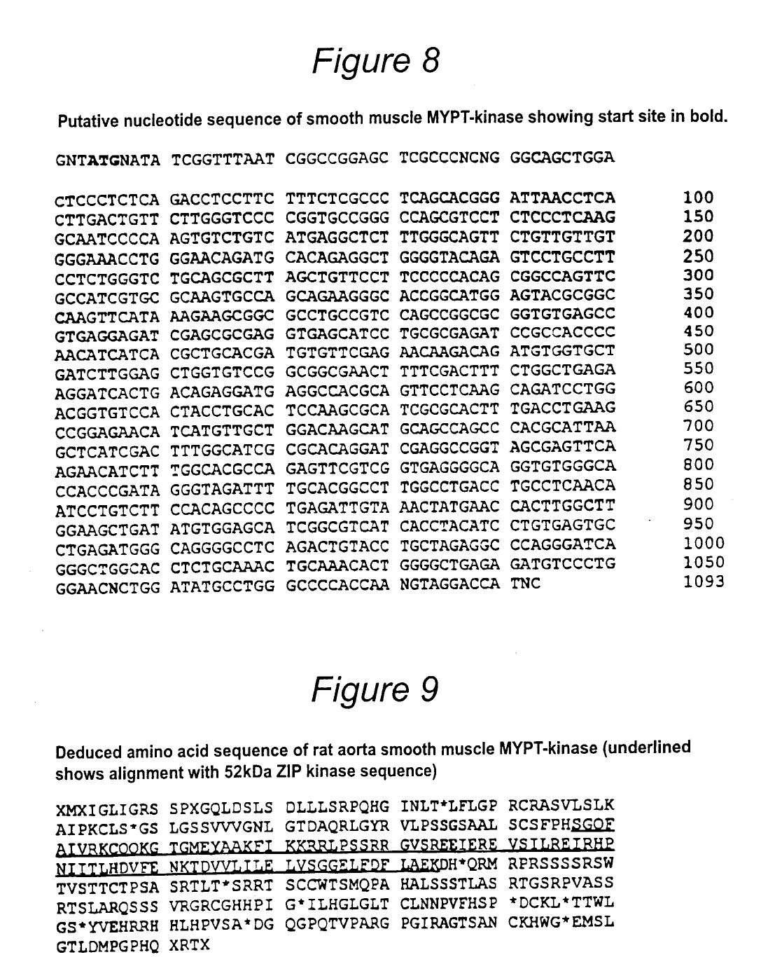

To identify the full length MYPT-kinase, a rat aorta smooth muscle cDNA library was screened with the I.M.A.G.E. dbEST AI660136 clone corresponding to the N-terminal region of ZIP kinase. The nucleotide sequence and conceptual translation of the putative smooth muscle MYPT-kinase is provided in FIGS. 8 and 9. As indicated below, possession of this full length clone allows the screening of compounds for their ability to act as specific modulators of this kinase activity.

Phosphorylation of Thr⁶⁹⁷ on full length MYPT1 in vitro by the native MYPT-kinase is considerably faster than by ROK. Interestingly, and in contrast to ROK, the MYPT-kinase more effectively phosphorylates full length MYPT1 at Thr⁶⁹⁷ than a C-terminal fragment (residues 514-963) of the protein containing this site. Inhibition of the native MYPT-kinase activity by the ROK inhibitor Y-27632 (Uehata et al, Nature 389:990-994 (1997)) occurs at levels that are 200-fold greater than that for ROK. Since Y-27632 is known to inhibit ROK in vivo and brings about decreased blood pressure in hypertensive mice (Sward et al, J. Physiol. 522:33-49 (2000)), the lack of sensitivity of SMPP1-1M kinase to the drug indicates that the enzyme participates in a Ca²⁺-sensitizing signal transduction pathway downstream of ROK. Significantly, the MYPT-kinase does not phosphorylate Ser⁸⁵⁴ on full length MYPT1. This contrasts with ROK, which has been reported to phosphorylate both Thr⁶⁹⁷ and Ser⁸⁵⁴ in vitro (Kawano et al, J. Cell. Biol. 147:1023-1038 (1999)). This finding indicates that Thr⁶⁹⁷ phosphorylation alone by the MYPT-kinase is sufficient to inhibit SMPP-1 activity. The MYPT-kinase, therefore, provides an excellent target on which to test anti-hypertensive drugs. Also, regulation of smooth muscle myosin phosphatase has broader implications for motility, migration and even metastasis in non-muscle cells which have a myosin II based component and contain myosin phosphatase, RhoGTPase, ROK and MYPT-kinase.

The I.M.A.G.E. dbEST AI660136 clone corresponding to the N-terminal region of ZIP kinase has been expressed as recombinant GST-fusion protein. This recombinant 38 kDa GST-rN-ZIP¹⁻³²⁰ kinase has been expressed in E. coli and found to be constitutively active and phosphorylate the Thr⁶⁹⁷ on the full length MYPT1 a rate equal to that of the native purified MYPT-kinase as well as demonstrating a similar insensitivity to Y-27632.

Experiments in which this rN-ZIPK was added to permealized rabbit longitudinal ileum smooth muscle strips demonstrate the Ca²⁺-sensitizing nature of the MYPT-kinase in vivo. A prior report demonstrated that full length ZIP-kinase could phosphorylate MLC20 in vitro (Hartshorne in Physiology of the Gastrointestinal Tract, ed. Johnson, L. R. (Raven Press, New York, N.Y.), pp. 423-482 (1987)). However, the present data indicate that in vivo, the MYPT-kinase does not lead to Ca²⁺-sensitization through the direct phosphorylation of MLC20 but by an inhibition of SMPP-1M activity through the phosphorylation of Thr⁶⁹⁷ on MYPT1. Administration of rN-ZIP¹⁻³²⁰ kinase to permeabilized ileam strips does not cause contraction in the absence of calcium as would be expected if indiscriminate phosphorylation of MLC20 was occurring. Instead, when rN-ZIP¹⁻³²⁰ kinase is added a 40% increase in muscular force is produced at the same submaximal calcium concentration. This defines Ca²⁺-sensitization and indicates that the MYPT provides a more specific pharmaceutical target in vascular hypertension than other upstream kinases (i.e., ROK).

In one embodiment, the present invention relates to a nucleic acid molecule that is at least 60%, 62%, 65%, 70%, 75%, 80%, 85%, 90%, 95%, 98% or more homologous to a nucleotide sequence (e.g., to the entire length of the nucleotide sequence) including the sequence shown in FIG. 8, or a complement thereof.

In a preferred embodiment, the isolated nucleic acid molecule includes the nucleotide sequence shown in FIG. 8 or complement thereof.

In another embodiment, the invention relates to a nucleic acid molecule that includes a nucleotide sequence encoding a protein having an amino acid sequence homologous to the amino acid sequence of FIG. 9. In a preferred embodiment, the nucleic acid molecule includes a nucleotide sequence encoding a protein having an amino acid sequence at least 50%, 55%, 60%, 65%, 70%, 75%, 80%, 81%, 85%, 90%, 95%, 98% or more homologous to an amino acid sequence including that shown in FIG. 9.

Another embodiment of the invention features nucleic acid molecules that specifically detect nucleic acid molecules that encode the amino acid sequence of FIG. 9 relative to nucleic acid molecules encoding unrelated proteins. For example, in one embodiment, such a nucleic acid molecule is at least 50, 100, 150, 200, 250, 300, 350, 400, 450, 500, 550, 600, 650, 700, 750, or 800 nucleotides in length and hybridizes under stringent conditions to a nucleic acid molecule comprising the nucleotide sequence shown in FIG. 8, or a complement thereof.

In other preferred embodiments, the nucleic acid molecule encodes a naturally occurring allelic variant of a polypeptide which includes the amino acid sequence of FIG. 9, wherein the nucleic acid molecule hybridizes to a nucleic acid molecule which includes the sequence of FIG. 8 under stringent conditions.

Another embodiment of the invention provides an isolated nucleic acid molecule which is antisense to a nucleic acid molecule that encodes the amino acid sequence shown in FIG. 9.

Another aspect of the invention provides a vector comprising a nucleic acid molecule as described above. In certain embodiments, the vector is a recombinant expression vector. In another embodiment, the invention provides a host cell containing a vector of the invention. The invention also provides a method for producing a protein of the invention by culturing in a suitable medium, a host cell, e.g., a mammalian host cell such as a non-human mammalian cell, containing a recombinant expression vector, such that the protein is produced.

Another aspect of this invention features isolated or recombinant proteins and polypeptides. In one embodiment, the isolated protein is the protein of FIG. 9. In a preferred embodiment, the protein has an amino acid sequence at least about 41%, 42%, 45%, 50%, 55%, 59%, 60%, 65%, 70%, 75%, 80%, 81%, 85%, 90%, 95%, 98% or more homologous to an amino acid sequence including that shown in FIG. 9.

Another embodiment of the invention features an isolated protein which is encoded by a nucleic acid molecule having a nucleotide sequence at least about 50%, 54%, 55%, 60%, 62%, 65%, 70%, 75%, 78%, 80%, 85%, 86%, 90%, 95%, 97%, 98% or more homologous to a nucleotide sequence (e.g., to the entire length of the nucleotide sequence) including the sequence of FIG. 8.

The proteins of the present invention or biologically active portions thereof, can be operatively linked to an unrelated polypeptide (e.g., heterologous amino acid sequences) to form fusion proteins. The invention further features antibodies, such as monoclonal or polyclonal antibodies, that specifically bind proteins of the invention. In addition, the proteins of the invention or biologically active portions thereof can be incorporated into pharmaceutical compositions, which optionally include pharmaceutically acceptable carriers.

In another aspect, the present invention provides a method for detecting the presence of a nucleic acid molecule, protein or polypeptide of the invention in a biological sample by contacting the biological sample with an agent capable of detecting a nucleic acid molecule, protein or polypeptide of the invention such that the presence of a nucleic acid molecule, protein or polypeptide of the invention is detected in the biological sample. In another aspect, the present invention provides a method for detecting the presence of a protein having the kinase activity of that of the invention in a biological sample by contacting the biological sample with an agent capable of detecting an indicator of the kinase activity such that the presence of kinase activity is detected in the biological sample.

In another aspect, the invention provides a method for modulating the kinase activity comprising contacting a cell capable of expressing the kinase of the invention with an agent that modulates the kinase activity such that the kinase activity in the cell is modulated. In one embodiment, the agent inhibits the kinase activity. In another embodiment, the agent stimulates the kinase activity. In one embodiment, the agent is an antibody that specifically binds to the kinase of the invention. In another embodiment, the agent modulates expression of the kinase by modulating transcription of a kinase gene or translation of a kinase mRNA of the invention. In yet another embodiment, the agent is a nucleic acid molecule having a nucleotide sequence that is antisense to the coding strand of the kinase mRNA or the kinase gene of the invention.

In one embodiment, the methods of the present invention are used to treat a subject having a disorder characterized by aberrant protein or nucleic acid expression or activity by administering to the subject an agent which is a modulator of the protein of the invention to the subject. In one embodiment, the modulator is a protein of the invention. In another embodiment the modulator is a nucleic acid molecule. In yet another embodiment, the modulator is a peptide, peptidomimetic, or other small molecule. In a preferred embodiment, the disorder characterized by aberrant protein or nucleic acid expression is a smooth muscle disorder.

In another embodiment, the present invention relates to methods for identifying compounds that can bind to the proteins of the invention and/or have a stimulatory or inhibitory effect on, for example, kinase expression or activity. Examples of such types of methods are described in U.S. Pat. No. 6,190,874. Further relevant details relating to other of the embodiments described above can also be found in U.S. Pat. No. 6,190,874 (including, for example, methods for determining percent homology, definitions of hybridization stringency conditions, methods of antibody production, types of expression vectors and host cells, types of formulations, etc.).

Certain aspects of the invention can be described in greater detail in the non-limiting Example that follows.

### EXAMPLE

### Experimental Details

Affinity purified anti-MYPT1 antibody was prepared by Quality Controlled Biochemicals Inc. Anti-ZIPK antibody was from Calbiochem. Gamma-linked ATP Sepharose was produced as described (Haystead et al, Eur. J. Biochem. 214:459-462 (1993)). Bovine brain ROK was a gift of Dr. Michael Walsh (University of Calgary). ROK inhibitor, Y-27632, was a gift from Dr. Yoshimura (Welfide Corp). Two recombinants based on the chicken MYPT1 isoforms (M130 and M133) were prepared as described (Ito et al, Biochemistry 36:7607-7614 (1997), Hirano et al. J. Biol. Chem. 272:3683-3691 (1997)). Thr⁶⁹⁷ substrate peptide, KKKRQSRRSTQGVTL, containing Arg⁶⁹⁰ to Lys⁷⁰¹ of MYPT1 was synthesized by Biomolecules Midwest. ³²P-Labelled myosin and glycogen phosphorylase a were prepared as described (Shirazi et al, J. Biol. Chem. 269:31598-31606 (1994)).

Kinase and phosphatase assays. Kinase assays included 10 μL of enzyme diluted in 25 mM Hepes, pH 7.4, 1 mM DTT, and 100 μM Thr⁶⁹⁷ peptide. Reactions were started with addition of 20 μL Mg²⁺ ATP (5 mM MgCl₂ and 0.1 mM ATP (5000 cpm/nmol) and carried out at 25° C. Reactions were terminated after 20 min with the addition of 100 μL of 20 mM H₃PO₄. Aliquots (100 μL) of the reaction mixture were spotted on to P81 paper and washed four times with 20 mM H₃PO₄. The P81 paper was placed into 1.5 mL Eppendorf tubes and ³²P incorporation was determined by scintillation counting. Phosphatase assays were carried out as described (Shirazi et al, J. Biol. Chem. 269:31598-31606 (1994)).

In-gel kinase assay. In gel kinase assays were performed as described (Kameshita et al, Anal. Biochem. 183:139-143 (1989)). Samples containing kinase activity were boiled (5 min) in sample buffer and separated in SDS-PAGE gels (10%) containing Thr⁶⁹⁷ peptide (0.5 mg/mL). After electrophoresis, the gels were incubated in 20% isopropanol containing 50 mM Hepes, pH 7.5 twice for 30 min, and washed in 50 mM Hepes, pH 7.5 containing 5 mM 2-mercaptoethanol. After denaturation with 6M guanidine-HCl, 5 mM 2-mercaptoethanol and 50 mM Hepes, pH 7.5, the kinases in the gels were renatured (5° C.) by incubation in successive dilutions of guanidine-HCL (3, 1.5, 0.75 and 0 M), 0.05% Tween-20, and 5 mM 2-mercaptoethanol for 45 min each. For the kinase reaction, the gels were equilibrated for 30 min in kinase buffer (50 mM Hepes, pH 7.5, 0.1 mM EGTA, 20 mM MgCl₂, and 2 mM DTT) prior to incubation with 25 μM [γ-³²P] ATP (1 μCi/μM). The reaction was terminated by washing the gels in 5% TCA/1% sodium pyrophosphate. The gels were dried and autoradiographed.

Purification of the SMPP-1M associated kinase. The SMPP-1M associated kinase was isolated from cow bladders following initial steps outlined for purification of SMPP-1M from pig bladder (Shirazi et al, J. Biol. Chem. 269:31598-31606 (1994)). Following extraction of the myofibrillar pellet, the extract was diluted with 2 volumes of buffer C (20 mM Tris, pH 7.5, 25 mM MgCl₂, and 1 mM DTT with protease inhibitors), clarified by centrifugation (100,000 g, 45 min) and applied to a 5.0×10-cm column of ethylenediamine γ-linked ATP Sepharose equilibrated in buffer C. The column was washed with buffer C, and then buffer C containing 100 μM geldanamycin to eliminate recovery of HSP90 (Fadden and Haystead submitted). Kinase activity was eluted in 5 ml fractions with 20 mM ATP in buffer C. Active fractions were pooled, dialyzed against buffer D (20 mM Tris, pH 8.0, 1 mM EDTA, 1 mM DTT) and applied to an AP-1Q anion exchange column (1.5×10-cm) equilibrated in buffer D. The column was washed with buffer D and developed with a 0-1M salt gradient. Fractions were assayed for SMPP-1M kinase activity. Active fractions were pooled, dialyzed against buffer E (20 mM Tris. pH 7.5, 10 mM MgCl₂, 1 mM DTT) and applied to an Cibicron Blue 3GA column (1.5×10-cm) equilibrated in buffer E. The column was developed with a 0-2M NaCl gradient; fractions containing SMPP-1M kinase activity were pooled, dialyzed against buffer D. Following concentration (2 ml) the pool was applied to a SMART Mono-Q PC 1.6/5 column. Fractions (50 μL) were assayed for SMPP-1M kinase activity. The purity of SMPP-1M kinase was assessed by SDS-PAGE and silver staining.

Mixed peptide sequencing. Fractions containing SMPP-1M kinase activity were separated by SDS-PAGE and electroblotted to PVM. The transferred proteins were stained with Amido Black and identified by mixed peptide sequences as described (Damer et al, J. Biol. Chem. 273:24396-24405 (1998)).

Preparation of recombinant GST-ZIPK fusion proteins. The GenBank dbEST database was searched with the complete sequence of human ZIPK. I.M.A.G.E. cDNA clones AI660136 (1-955 bps) and AW237698 (19-930 bps) encoding the N-terminal⁽¹⁻³²⁰⁾ portion of ZIPK were obtained from Genome Systems Inc. Both clones are 99.9% homologous to the N-terminal domain of human ZIPK (Inbal et al, Mol. Cell. Biol. 20:1044-1054 (2000)). cDNA clones were in-frame inserted into vector pGEX-4T-1 (Pharmacia) in order to express the glutathione S-transferase (GST) fusion protein. E. coli cells were cultured in LB broth, 50 μg/mL ampicillin, overnight at 37° C. Cells were induced with 100 μM isopropyl-β-D-thiogalactopyranoside, and GST-ZIK isolated using glutathione-Sepharose 4B beads.

Immunoprecipitation techniques. For ZIP-like-kinase co-immunoprecipitation experiments, tissue homogenates (1:5 w/v) from rabbit bladder were prepared in 25 mM Hepes, pH 7.5, 0.1 mM EGTA, 0.1 mM EDTA, 1 mM DTT, 0.5% Triton X-100, 600 mM NaCl and protease inhibitors. Homogenates were centrifuged for 10 min (10,000×g); the supernatant was removed, diluted 5-fold with buffer A, and precleared with protein A Sepharose beads (1 hr at 5° C.). Tissue extract was incubated overnight with 10 μg rabbit polyclonal anti-ZIPK, followed by harvest with protein A Sepharose. Immunoprecipitated proteins were resolved by SDS-PAGE, transferred to PVDF membrane and immunoblotted with rabbit anti-MYPT1 antibody. The membranes were developed using ECL (Pharmacia). For MYPT1 co-immunoprecipitation experiments, tissue homogenates from rabbit bladder were prepared as detailed above. The extract was incubated overnight with 10 μg rabbit polyclonal anti-MYPT1, followed by harvest with protein A Sepharose. SDS-PAGE and ZIPK immunoblots were performed as above.

[³²P] orthophosphate labeling of rabbit bladder. Rabbit bladder was removed from rabbits anaesthetized with halothane according to approved protocols. Two groups of intact smooth muscle sheets (8 mm×8 mm) were incubated in Hepes-buffered Krebs solution in the presence of [³²P] PO₄³⁻ (5 mCi/mL) at 25° C. for 1 hour. To inhibit endogenous phosphatase activity muscle pieces were treated first with calyculin A (10 μM), then vehicle (control) or carbachol (50 μM) for a further 15 minutes. The tissues were flash frozen in liquid N₂ then homogenized in lysis buffer (20 mM Tris-HCl, pH 7.5, 250 mM sucrose, 5 mM EDTA, 1 mM DTT, 10 nM microcystin, 2 μg/mL aprotinin, 2 μg/mL leupeptin, and 0.1 mM PMSF) and centrifuged (20,000×g). The pellets were extracted with buffer B, centrifuged and fractionated by micro anion-exchange chromatography using a SMART FPLC (Pharmacia). Column fractions were assayed for ZIPK activity.

Results

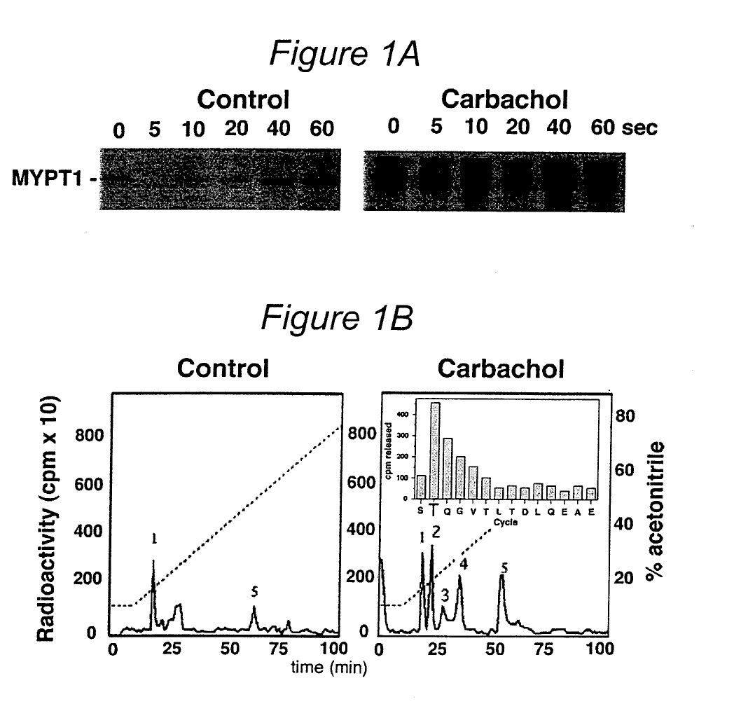

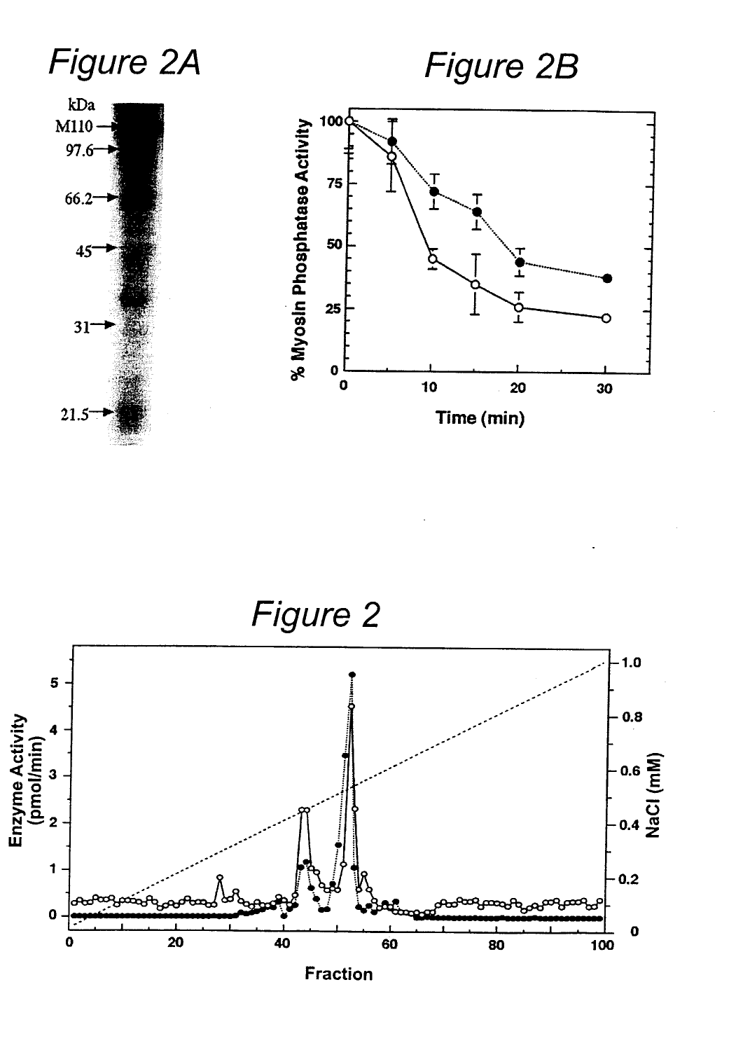

Identification of MYPT1 Phosphorylation sites in Response to Ca²+-Sensitization. Through ³²P-labeling of intact smooth muscle, four phosphopeptides on MYPT1 were identified whose phosphorylation state was increased in response to Ca²⁺-sensitizing agents such as carbachol (FIGS. 1a and 1b). Phosphopeptide mapping and peptide sequencing identified the major carbachol sensitive site as Thr⁶⁹⁷ on MYPT1 (FIGS. 1b and 1c). Furthermore, the presence of an endogenous MYPT1 kinase that copurifies and phosphorylates Thr⁶⁹⁷ was confirmed, inactivating SMPP-1M in vitro (FIG. 2).

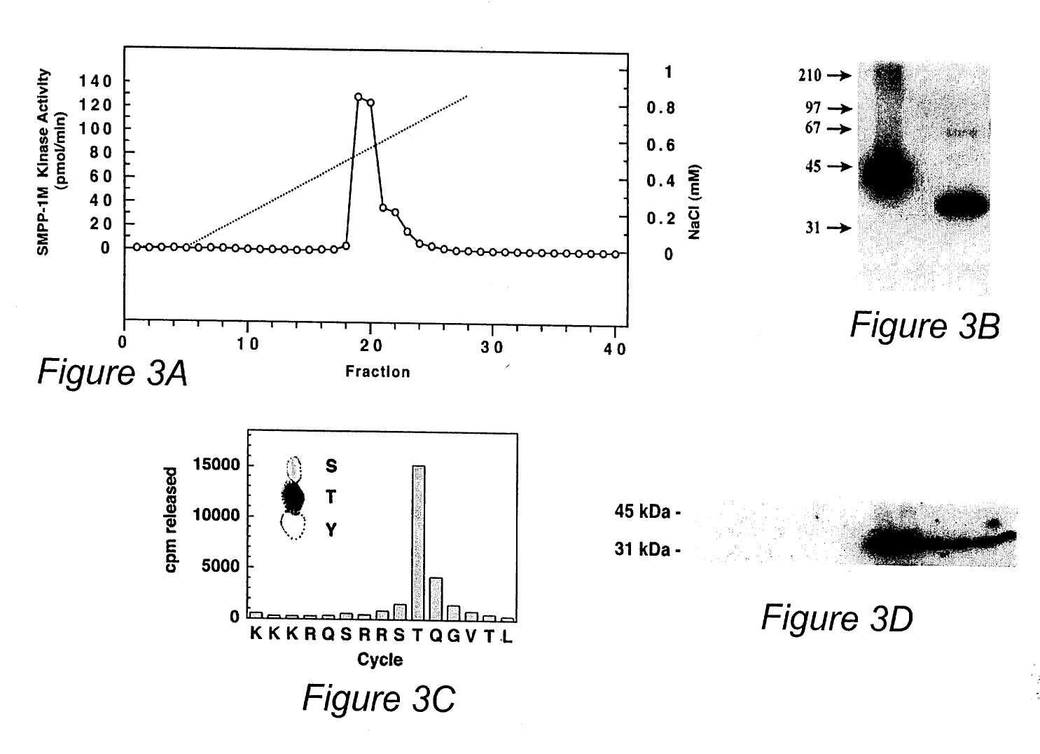

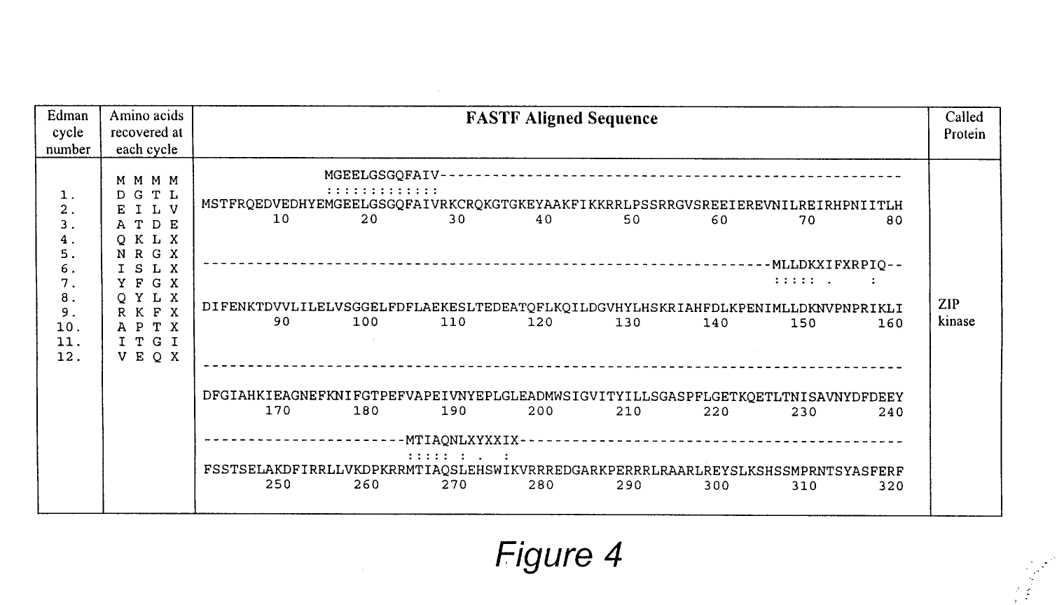

Purification and Identification of the Endogenous MYPT1 Kinase. To identify the endogenous kinase that is copurified with MYPT1 (FIG. 2), a substrate peptide with sequence corresponding to the Thr⁶⁹⁷ phosphorylation site of MYPT1 was synthesized. Kinase activity was isolated from the myofibrilar pellet of cow bladder and purified to near homogeneity using a γ-phosphate linked ATP-Sepharose affinity column. A single band of kinase activity toward the Thr⁶⁹⁷ peptide was identified by an in-gel kinase (1 and 2D SDS-PAGE) assay (Kameshita et al, Anal. Biochem. 183:139-143 (1989)) at 32 kDa (FIG. 3). An identical band of kinase activity was obtained using an in-gel kinase assay and the C-terminal fragment of MYPT1 as the substrate. The SMPP-1M kinase at 32 kDa in the gels was identified by mixed peptide sequencing and was most similar to HeLa zipper interacting protein kinase ZIP kinase (ZIPK) (FIG. 4). Further in gel kinase analysis by 2D SDS PAGE and mixed peptide sequencing confirmed that the 32 kDa band contained a single protein and not any other protein kinase. A previous report (Kawai et al, Mol. Cell. Biol. 18:1642-1651 (1998)) on full-length mammalian ZIPK indicated masses of 51.4 kDa and 52.5 kDa for the mouse and human isoforms, respectively, as compared to a mass of 32 kDa for the SMPP-1M-associated kinase identified herein. Whether the latter is a proteolyzed fragment of full length ZIPK or is a smaller smooth muscle specific isozyme remains to be determined. Preliminary Western blotting experiments with ZIPK antibody indicate the presence of two bands of approximately both 58 kDa and 34 kDa in most rat smooth muscles tested. Based on these studies the SMPP-1M associated kinase identified herein is referred to as “ZIP-like-kinase”.

The enzymatic properties of native (ZIP-like) and recombinant ZIPK were investigated in vitro. Recombinant 38 kDa ZIPK was expressed in E. coli and found to be constitutively active and phosphorylate the Thr⁶⁹⁷ peptide and full length MYPT1 at Thr⁶⁹⁷ at a rate equal to that of the native purified protein.

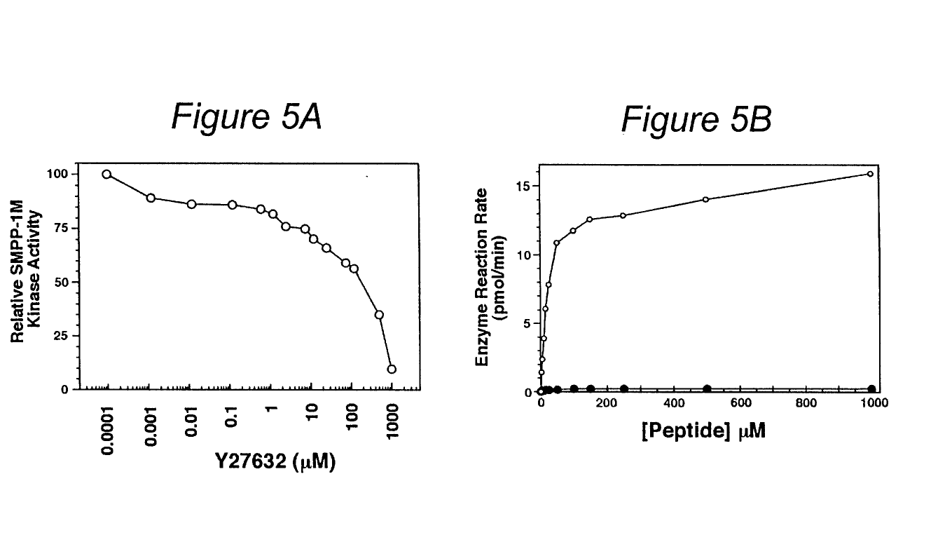

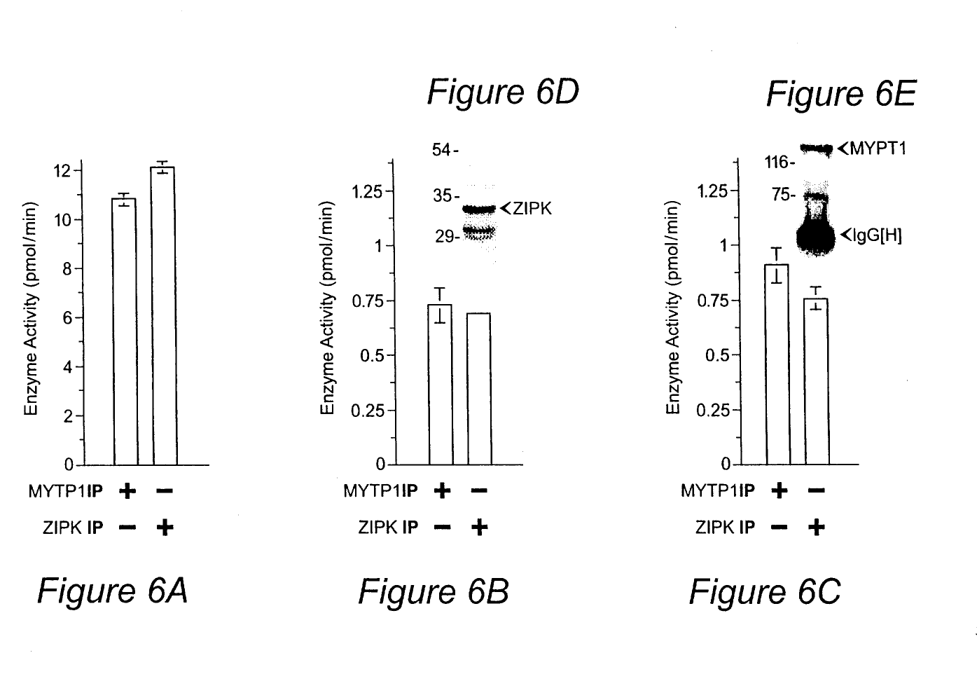

FIG. 5 shows that inhibition of native ZIP-like-kinase by the ROK inhibitor Y-27632 (Uehata et al, Nature 389:990-994 (1997)) occurs at levels that are 200-fold greater than that for ROK. Recombinant ZIPK demonstrated a similar insensitivity to Y-27632. Since Y-27632 is known to inhibit ROK in vivo and brings about decreased blood pressure in hypertensive mice, the lack of sensitivity of ZIP-like-kinase to the drug may suggest that the enzyme participates in a Ca²⁺ sensitizing signal transduction pathway downstream of ROK (Uehata et al, Nature 389:990-994 (1997)). Phosphorylation of the Thr⁶⁹⁷ peptide and full length MYPT1 (rM133) in vitro by native ZIP-like-kinase was considerably faster than by ROK (about 15-fold FIG. 5). Interestingly, and in contrast to ROK, ZIP-like-kinase more effectively phosphorylated full length MYPT1 at Thr⁶⁹⁷ than a C-terminal fragment (residues 514-963) of the protein containing this site (FIG. 5). Recombinant ZIPK displayed identical properties. Significantly, ZIP-like-kinase or ZIPK did not phosphorylate Ser⁸⁵⁴ on full length MYPT1. This contrasts with ROK, which has been reported to phosphorylate both Thr⁶⁹⁷ and Ser⁸⁵⁴ in vitro (Kawano et al, J. Cell. Biol. 147:1023-1038 (1999)). This finding indicates that Thr⁶⁹⁷ phosphorylation alone is sufficient to inhibit SMPP-1M activity. To characterize recombinant ZIPK further, the sites of auto phosphorylation on the enzyme were determined. FIG. 5 also shows the sequence and identifies S¹¹⁰ and T¹¹² as phosphorylated residues in the activation loop. This finding suggests two phosphorylation events are required to activate ZIPK. Importantly similar analysis on ZIP-like-kinase immunoprecipitated from ³²P labeled bladder showed activation correlated with increased phosphorylation (see below, FIG. 7).

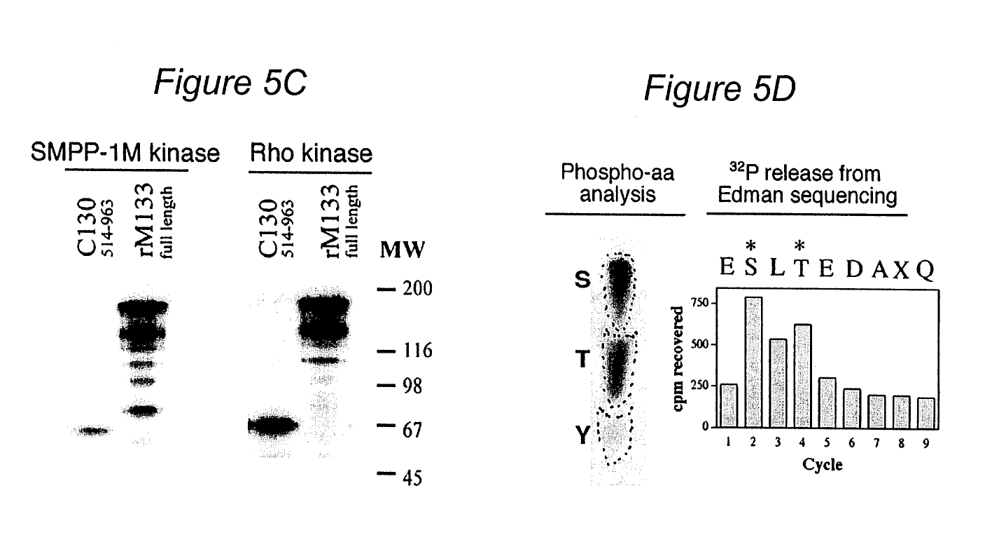

ZIP kinase and MYPT1 are colocalized in smooth muscle. Although, SMPP-1M and ZIP-like-kinase co-purified through three distinct chromatography steps (FIGS. 2 and 3), immunoprecipitation was employed to determine whether ZIP-like-kinase and MYPT1 interact in smooth muscle. Immunoprecipitates of MYPT1 from rabbit bladder contained ZIPK as evidenced from immunoblotting, and similarly, when ZIP-like-kinase was immunoprecipitated, MYPT1 was detected by immunoblotting (FIG. 6). ZIP-like-kinase activity in MYPT1 immunoprecipitates was also measured using the Thr⁶⁹⁷ peptide substrate by in vitro assay and by in-gel kinase assay. Kinase activity was recovered from both anti-MYPT1 and anti-ZIPK immunoprecipitates. SMPP-1M phosphatase activity in the immunoprecipitates was measured against two known SMPP-1M substrates, myosin and glycogen phosphorylase a (Shirazi et al, J. Biol. Chem. 269:31598-31606 (1994)). SMPP1-1M phosphatase activity was present in the ZIP-like-kinase and MYPT1 immunopellets. These experiments demonstrate that an active ZIP-like-kinase is associated with fully functional SMPP-1M phosphatase in smooth muscle.

ZIP-like-kinase is phosphorylated and activated in vivo by carbachol. To determine the mechanism of activation of ZIP-like-kinase in vivo the protein was immunoprecipitated from ³²P-labeled rabbit bladders following treatment with the Ca²⁺ sensitizing drug carbachol. Treatments were carried out in the presence of calyculin A (an inhibitor of type 1 and 2A protein phosphatases) to inhibit endogenous ZIP-like-kinase phosphatase activity. FIG. 7 shows that ZIP-like-kinase was phosphorylated and activated in rabbit bladder smooth muscle by exposure to carbachol. In experiments carried out in the absence of calyculin A the activation of ZIP-like-kinase was reduced by about 50% indicating control of the kinase via a kinase/phosphatase couplet (FIG. 7). Phospho amino acid analysis of immunoprecipitated ZIP-like-kinase from ³²P-labeled bladder identified the presence of both phosphoserine and phosphothreonine. Preliminary in vitro experiments suggest that ROK does not directly phosphorylate ZIP-like-kinase indicating that additional components (such as a ZIP-like-kinase kinase) may be required. Consistent with this hypothesis treatment of carbachol and calyculin A treated bladder with Y-27632 (10 μM) caused a significant inhibition of ZIP-like-kinase activity (FIG. 7).

All documents cited above are hereby incorporated in their entirety by reference.

What is claimed is:

1. An isolated nucleic acid encoding MYPT kinase.

2. The nucleic acid according to claim 1 wherein said MYPT kinase is mammalian MYPT kinase.

3. The nucleic acid according to claim 1 wherein said MYPT kinase has the amino acid sequence set forth in FIG. 9.

4. An isolated nucleic acid encoding mammalian MYPT kinase, or portion thereof of at least 15 consecutive bases, or complement thereof.

5. The isolated nucleic acid according to claim 4 wherein the nucleic acid encodes the amino acid sequence set forth in FIG. 9, or portion thereof of at least 5 amino acids.

6. The isolated nucleic acid according to claim 5 wherein the nucleic acid has the sequence shown in FIG. 8, or a sequence substantially identical thereto, or portion thereof of at least 15 consecutive bases.

7. The isolated nucleic acid according to claim 6 wherein said nucleic acid has the sequence shown in FIG. 8, or portion thereof of at least 15 consecutive bases.

8. The isolated nucleic acid according to claim 7 wherein the nucleic acid has the sequence shown in FIG. 8.

9. A recombinant molecule comprising said nucleic acid according to claim 1 and a vector.

10. The recombinant molecule according to claim 9 further comprising a promoter operably linked to said nucleic acid sequence.

11. A host cell comprising said recombinant molecule according to claim 9.

12. A method of producing MYPT kinase comprising culturing said host cell according to claim 11 under conditions such that said nucleic acid sequence is expressed and said MYPT kinase is thereby produced.

13. A recombinant molecule comprising the nucleic acid sequence according to claim 4 and a vector.

14. The recombinant molecule according to claim 13 further comprising a promoter operably linked to said nucleic acid sequence.

15. A host cell comprising said recombinant molecule according to claim 13.

16. A method of producing mammalian MYPT kinase, or portion thereof, comprising culturing said host cell according to claim 15 under conditions such that said nucleic acid sequence is expressed and said mammalian MYPT kinase, or portion thereof, is thereby produced.

17. An isolated mammalian MYPT kinase or portion thereof of at least 5 consecutive amino acids.

18. The protein according to claim 17 wherein said protein has the amino acid sequence shown in FIG. 9.

19. An antibody specific for the protein, or portion thereof, of claim 17.

20. A method of screening a test compound for anti-hypertensive activity comprising contacting MYPT kinase with MYPT1, or portion thereof comprising Thr⁶⁹⁷,in the presence and absence of said test compound and determining the ability of said compound to modulate the phosphorylation of Thr⁶⁹⁷ by said kinase.

21. A kit for use in the detection of MYPT kinase comprising a compound that specifically binds to MYPT kinase disposed within a container means.

22. The kit according to claim 21 wherein said compound is an antibody or binding fragment thereof.
